(19) United States
(12) Patent Application Publication
(10) Pub. No.: US 2008/0171885 A1
(43) Pub. Date: Jul. 17, 2008

(54) Process for Preparation of Highly Pure Trandolapril

(75) Inventors: Girij Pal Singh, Pune (IN); Mukesh Jagannath Wani, Pune (IN); Hemraj Mahadeorao Lande, Pune (IN); Adinath Murlidhar Jain, Pune (IN)

Correspondence Address: MERCHANT & GOULD PC, P.O. BOX 2903, MINNEAPOLIS, MN, 55402-0903, US

(73) Assignee: Lupin Limited, Mumbai (IN)

(21) Appl. No.: 11/816,251

(22) PCT Filed: Sep. 6, 2005
(86) PCT No.: PCT/IN2005/000301

§ 371 Date: Aug. 14, 2007

(30) Foreign Application Priority Data
Feb. 14, 2005 (IN) 155/MUM/2005

(51) Int. Cl. C07D 209/26

(52) U.S. Cl. 548/501

(57) ABSTRACT

The present invention provides an improved process for preparation of highly pure trandolapril. The process comprises of the following steps: (i) crystallization of mixture of racemic benzyl trans-(2S, 3aR, 7aS)-octahydro-1H-indole carboxylate p-toluene sulphonic acid salt (IIa.p-TsOH) and benzyl trans-(2R, 3aS, 7aR)-octahydro-1H-indole carboxylate p-toluene sulphonic acid salt (IIb.p-TsOH) through appropriate selection of solvents to enrich the purity to >99% from a mixture containing the other diastereomers (IIc-h.p-TsOH) up to 6%, (ii) optical resolution of racemic mixture of benzyl trans-(2S, 3aR, 7aS)-octahydro-1H-indole carboxylate (Na) and benzyl trans-(2R, 3aS, 7aR)-octahydro-1H-indole carboxylate (lib) with (−)-dibenzoyl-L-tartaric acid monohydrate in an appropriately selected solvents and temperature, (iii) reaction of benzyl ester Ma with N-[1-(S)-ethoxycarbonyl-3-phenylpropyl]-(S)-alanine N-carboxy anhydride (III a, NEPA-NCA hereafter) to get trandolapril benzyl ester (IVa), and finally (iv) crystallization of crude trandolapril from appropriate solvents.

### FIELD OF THE INVENTION

The present invention relates to process for manufacturing trandolapril of formula I of high enantiomeric purity.

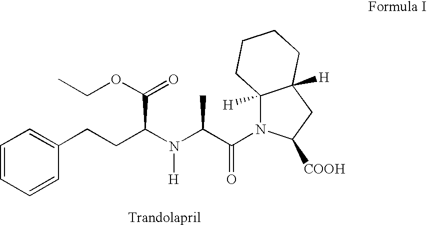

### BACKGROUND OF THE INVENTION

Trandolapril [CAS Reg. No. [87679-37-6]], chemically known as N-(1(S)-carboethoxy-3-phenylpropyl)-S-alanyl-(2S, 3aR, 7aS)-octahydroindole-2-carboxylic acid, was first disclosed in U.S. Pat. No. 4,933,361. Trandolapril is a well-known antihypertensive agent due to its Angiotensin Converting Enzyme (ACE) inhibitory activity.

U.S. Pat. No. 4,933,361 describes the synthesis of trandolapril that employs racemic (2S, 3aR, 7aS)-trans-octahydro-1H-indole-2-carboxylic acid (Ia) and (2R, 3aS, 7aR)-trans-octahydro-1H-indole-2-carboxylic acid (Ib) as intermediate.

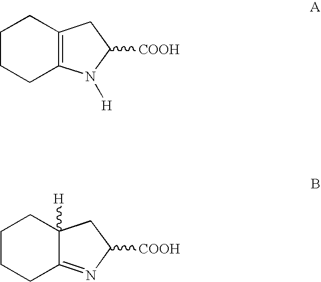

U.S. Pat. No. 4,933,361 discloses several methods for the preparation of the above mentioned octahydro-1H-indole-2-carboxylic acids (Ia-h). Such methods for preparation of trans octahydro-1H-indole-2-carboxylic acids (Ia-d) employ the reduction of the mixture of enamine of the formula (A) and imine of formula (B) by catalytic hydrogenation using Raney Nickel, or Pt/C in glacial acetic acid or reduction with complex borohydrides or borane-amine complexes. However these methods are commercially non-viable since the undesired cis isomers (Ie-h) are produced in major amount (i.e. more than 60%).

In copending application No. 1033/MUM/2003 there is disclosed and claimed an improved method for the production of desired racemic trans octahydroindole-1H-2-carboxylic acids (Ia and Ib) by the reduction of mixture of enamine compound formula (A) and imine compound of formula (B) using Rh/C under alkaline condition in presence of water and water miscible organic solvent.

This method provided diastereomeric mixture of octahydroindole-1H-2-carboxylic acids (Ia-h) in which the ratio of trans acids (Ia-Ih) to cis acids (Ie-Ih) was greater than or equal to 1:1. In the subsequent process the mixture of acids (Ia-h) was enriched to >94% racemate of trans octahydroindole-1H-2-carboxylic acids (Ia and Ib) by selective fractional crystallization initially from isopropanol and then from methanol. The resulting racemate of trans exo amino acids (Ia and Ib) was >94% containing <1% of the trans endo isomers (Ic and Id); and <5% of the cis isomers (Ie-h). The composition of cis and trans acids in the mixture was determined by converting the mixture to benzyl esters (IIa-h) and then checking the purity of benzyl ester by HPLC method.

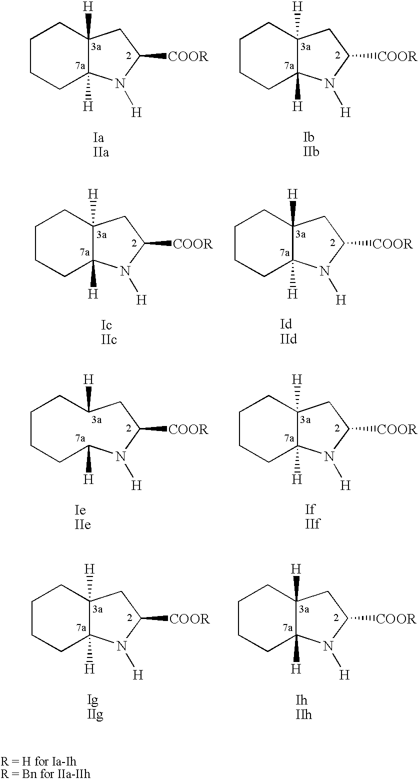

The synthesis described in U.S. Pat. No. 4,933,361 is shown in scheme 1 which involves conversion of racemic trans acids Ia and Ib to corresponding mixture of hydrochloride salts IIa.HCl and IIb.HCl with benzyl alcohol and thionyl chloride. The mixture of hydrochloride salts IIa.HCl and IIb.HCl was neutralised with N-methyl morpholine in dimethyl formamide to give racemic mixture of free benzyl esters IIa and IIb which was condensed with N-[1-(S)-ethoxycarbonyl-3-phenylpropyl]-(S)-alanine (NEPA, IIIb) by using 1-hydroxybenzotriazole and dicyclohexylcarbodiimide to obtain a diastereomeric mixture of trandolapril benzyl esters IVa and IVb.

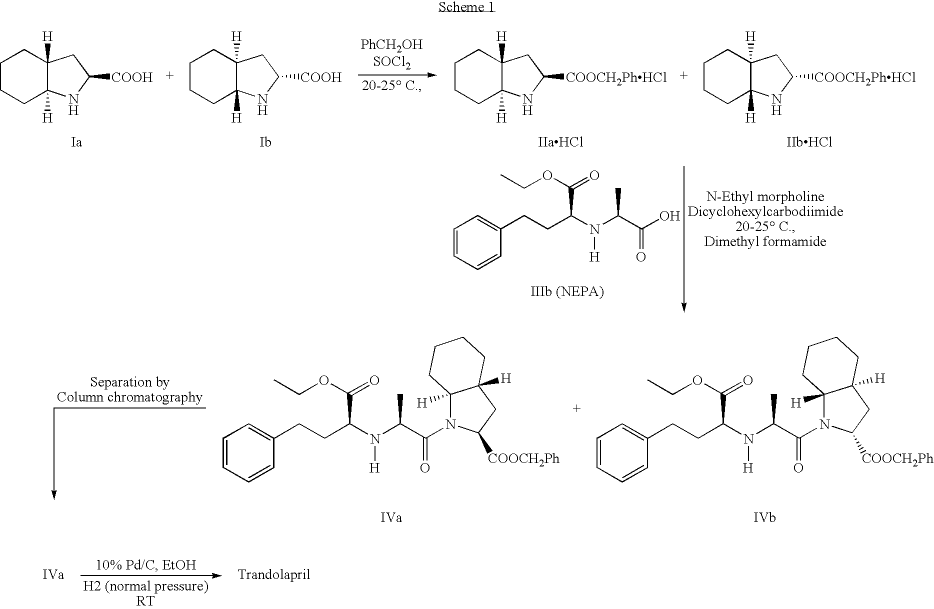

The diastereomers IVa and IVb were separated by column chromatographic method to obtain pure isomer IVa which was then subjected to hydrogenolysis with 10% Pd/C in ethanol to afford trandolapril as a foamy material.

The method described in U.S. Pat. No. 4,933,361 suffers from the several drawbacks such as:
i) it gives very low yield of required trans acids Ia and Ib,
ii) it requires separation of trandolapril benzyl ester (IVa) from its diastereomer IVb by column chromatography which is not suitable for large-scale production, and
iii) it provides trandolapril as foamy solid that is difficult to isolate.

U.S. Pat. No. 6,335,453 assigned to Kaneka Corporation discloses a general method for preparation of N-[1-(S)-ethoxycarbonyl-3-phenylpropyl]-(S)-alanyl-amino acids (IIIc) having low content of diketopiperazine (IIId) which involve reaction of corresponding amino acid with NEPA-NCA (IIIa) under basic condition at pH 9-12 in aqueous medium or in biphasic medium consisting mixture of organic solvent and water in the ratio 96:4 to 0:100. In this method at least 2 molar equivalent of amino acid is used. Moreover, we found that trandolapril prepared by following this method was contaminated with NEPA (IIIb) which was formed presumably by hydrolysis of NEPA-NCA (IIIa). Thus, the method disclosed in U.S. Pat. No. 6,335,453 B1 suffers from the following disadvantages:
i) it requires at least 2 molar equivalent of amino acid which increases the cost, and
ii) it provides trandolapril contaminated with NEPA (IIIb)

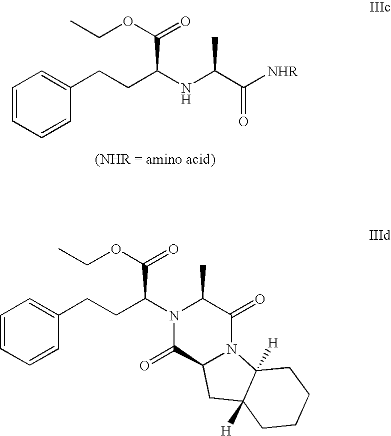

The resolution of the racemic benzyl esters IIIa and IIb is disclosed in Drug Design and Discovery, 1992, vol 9, pp 11-28 by using DBTA. The DBTA precipitates the salt of benzyl (2S, 3aR, 7aS)-trans-octahydro-1H-indole-2-carboxylate (IIa.DBTA) which is the required one for synthesis of trandolapril. As described in this publication, the resolution is achieved by treating the racemic benzyl esters IIa and IIb with DBTA in absolute ethanol followed by crystallization of crude solid from ethanol. It was found that by following this method of preparation of pure enantiomer IIa, transesterification of the benzyl ester takes place leading to the formation of undesired ethyl ester (IIj). The formation of salt IIj.DBTA was revealed from the mass spectrum which showed a peak at m/z 197 amu (M+1) arising from ethyl ester IIj. It was also found that when resolution and crystallization was carried out in methanol as solvent then the transesterification of the benzyl ester leading to the formation of undesired methyl ester (IIi) occurs.

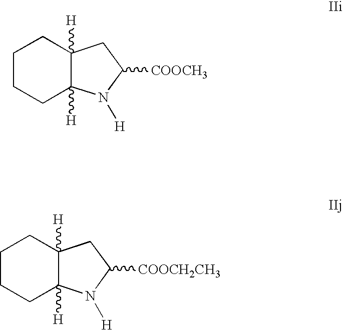

This was evident from the fact that trandolapril manufactured from enantiomer IIa obtained by following the method of resolution as described in above publication had the contamination of the trandolapril ethyl ester (IVj) as indicated by peak at m/z 459.3 amu (M+1) (when ethanol was used for resolution and recrystallization). Similarly, trandolapril methyl ester (IVi) as indicated by peak at m/z 445 amu (M+1) was formed when methanol was used as solvent for resolution and recrystallization. These impurities were detected by their mass spectra were formed in the range of 5-12% as per HPLC analysis. The removal of these trandolapril methyl ester (IVi) or trandolapril ethyl ester (IVj) impurities from trandolapril resulted in significant loss in yield.

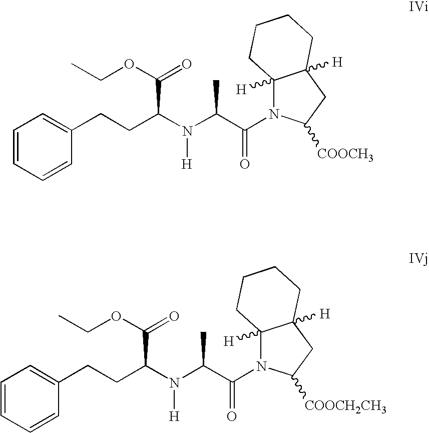

Thus, the resolution method described in Drug Design and Discovery, 1992, vol 9, pp 11-28 suffers from the disadvantage of undergoing side reaction i.e. transesterification of benzyl ester which complicates the subsequent steps and finally leads to contamination of impurities in the trandolapril that are arising from the transesterification products.

It is an object of the present invention to solve the problem of transesterification and provide a process for the preparation of highly pure trandolapril of Formula I which is simple and industrially suitable process and which can provide trandolapril in very high purity (i.e. >99%).

It is a further object of the present invention to provide a process for preparation of highly pure trandolapril of Formula I which is cost effective and also easy to operate on plant scale.

The applicants have found that the problem of transesterification may be solved by carrying out the resolution of racemic benzyl esters IIa and IIb in aprotic solvent selected from dimethyl formamide, dimethyl sulphoxide, acetonitrile or a mixture thereof.

### SUMMARY OF THE INVENTION

A process for the preparation of highly pure trandolapril of Formula I

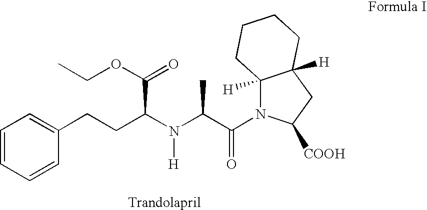

comprising the steps of:
a) enriching a racemic mixture of benzyl trans (2S, 3aR, 7aS)-octahydro-1H-indole-2-carboxylate p-toluene sulphonic acid salt (IIa.p.TsOH) and benzyl trans (2R, 3aS, 7aR)-octahydro-1H-indole-2-carboxylate p-toluene sulphonic acid salt (IIb.p-TsOH) to more than 99% from a mixture containing the other diastereomers (IIc-h.p-TsOH) up to 6%,
b) converting the mixture of the said salts IIa.p-TsOH and IIb.p-TsOH to corresponding mixture of free bases benzyl trans (2S, 3aR, 7aS)-octahydro-1H-indole-2-carboxylate (IIa) and benzyl trans (2R, 3aS, 7aR)-octahydro-1H-indole-2-carboxylate (IIb),

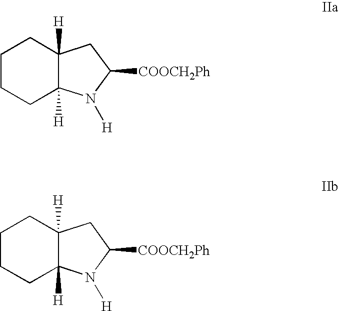

c) optically resolving the racemate benzyl trans (2S, 3aR, 7aS)-octahydro-1H-indole-2-carboxylate (IIa) and benzyl trans (2R, 3aS, 7aR)-octahydro-1H-indole-2-carboxylate (IIb) with (−)-dibenzoyl-L-tartaric acid monohydrate (DBTA hereafter) to obtain pure enantiomer Iia,
d), reacting benzyl ester IIa with N-[1-(S)-ethoxycarbonyl-3-phenylpropyl]-(S)-alanine N-carboxy anhydride (IIIa) to prepare trandolapril benzyl ester,

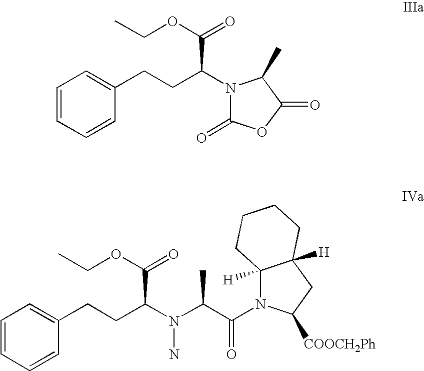

e) converting trandolapril benzyl ester (IVa) to crude trandolapril by hydrogenolysis, and
f) crystallizing crude trandolapril from the mixture of ethanol-diisopropyl ether to yield pure trandolapril (>99%).

According to a preferred aspect of the invention there is provided a process for the preparation of highly pure trandolapril of Formula I comprising the following steps:
a. converting octahydroindole-1H-2-carboxylic acids (Ia-h) to corresponding benzyl ester p-toluene sulphonic acid salts (IIa-h. p-TsOH) by the reaction of benzyl alcohol and p-toluene sulphonic acid monohydrate in refluxing cyclohexane and simultaneously removing the water formed during reaction by azeotropic distillation,
b. distilling out cyclohexane under reduced pressure and stirring the residue in diisopropyl ether,
c. filtering the solid and drying under reduced pressure,
d. heating mixture of salts IIa-h.p-TsOH in mixture of dichloromethane and cyclohexane to reflux temperature,
e. addition of extra quantity of cyclohexane at reflux temperature,
f. continuing reflux for some period of time, and
g. crystallizing of the mixture of IIa.p-TsOH and IIb.p-TsOH at 25-30° C. and followed by filtration of the same.

According to a further preferred aspect there is provided step of resolution comprises the following steps:
a. conversion of p-toluene sulphonate salts IIa.p-TsOH and IIb.p-TsOH to mixture of racemic esters IIa and IIb,
b. preparing solution of racemic mixture of IIa and IIb in acetonitrile,
c. cooling the solution to 15-20° C.,
d. dilution with dimethyl formamide,
e. addition of solution of DBTA at 15-20° C.,
f. optionally seeding with salt IIa.DBTA,
g. stirring at 15-20° C. for 4-5 hours for crystallization of DBTA salt of pure enantiomer IIa (IIa.DBTA), and
h. filtration and washing of salt IIa.DBTA with acetonitrile.

According to still further aspect of the invention the step of recrystallization of crude trandolapril comprises of the following steps:
a. dissolving crude trandolapril in mixture of ethanol-diisopropyl ether (2:5) by heating to reflux temperature;
b. continuing reflux for 10-15 minutes;
c. cooling the solution to 25-30° C.; and
d. crystallizing at 25-30° C. followed by filtration and washing with diisopropyl ether.

### DETAILED DESCRIPTION OF THE INVENTION

The present invention has four parts as shown below in scheme 2.

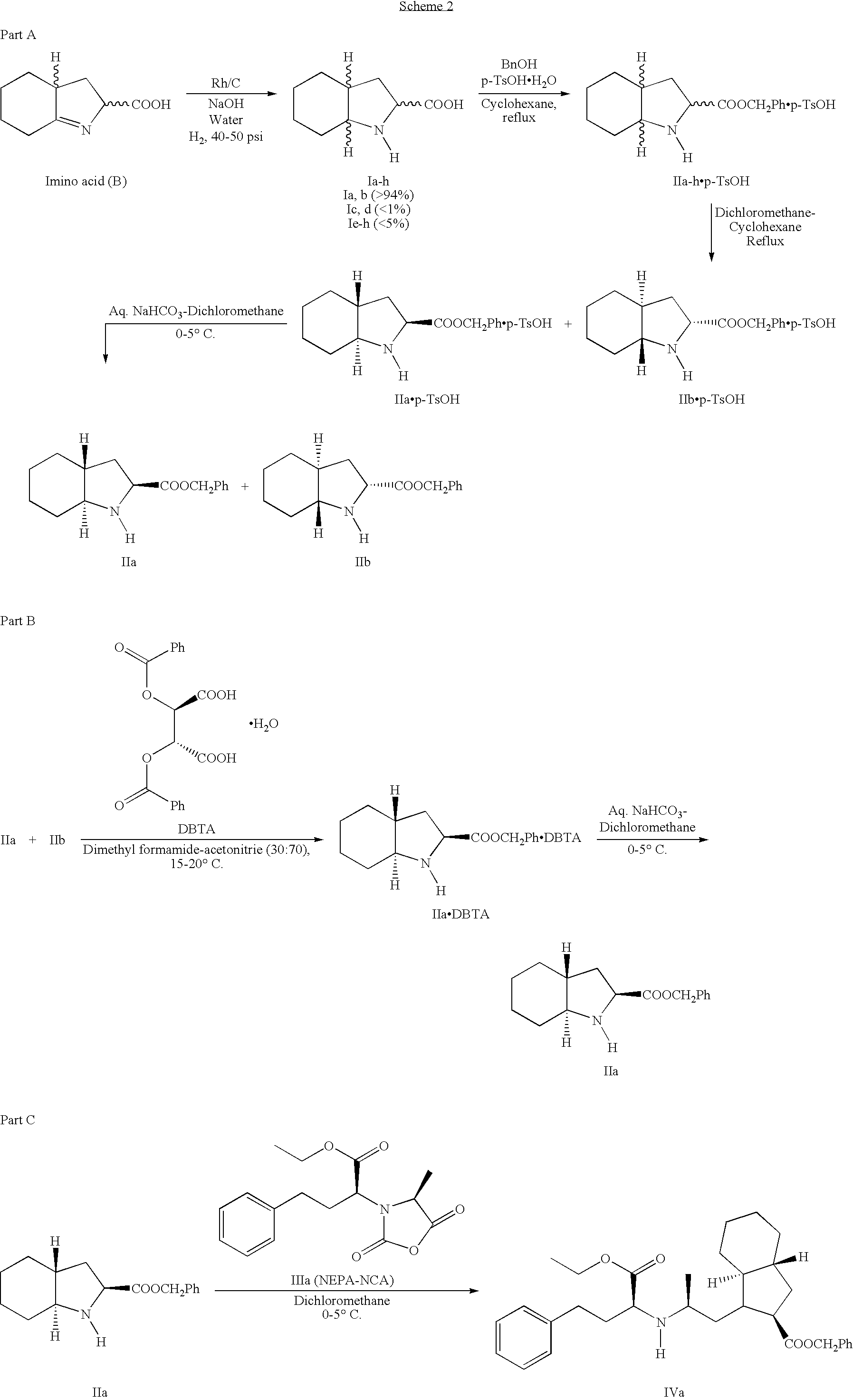

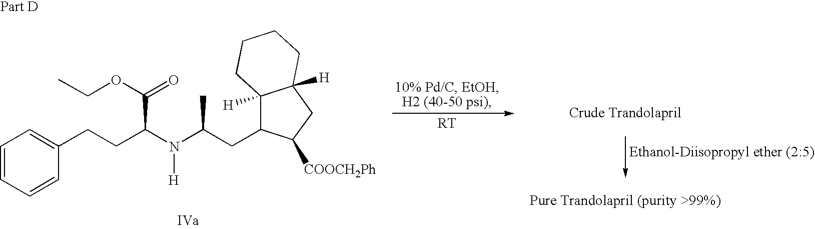

Racemic trans octahydroindole-1H-2-carboxylic acids (Ia and Ib) were prepared as per process described in the copending application No. 1033/MUM/2003 by the reduction of mixture of enamine compound formula (A) and imine compound of formula (B) using Rh/C under alkaline condition in presence of water and water miscible organic solvent. The purity of racemate of trans exo amino acids (Ia and Ib) was >94% and it contain <1% of the trans endo isomers (Ic and Id); and <5% of the cis isomers (Ie-h). The purification of trans exo acids (Ia and Ib) up to 99% was achieved after repeatedly crystallization from methanol but the yield was poor and hence this method of purification was not commercially feasible.

The process for enriching the p-toluene sulphonic acid salts of IIa and IIb to >99% purity is achieved by the present invention. The octahydroindole-1H-2-carboxylic acid (Ia-h) containing >94% of the trans racemate Ia and Ib; <1% of the trans isomers (Ic) and (Id); and <5% of the cis-diasteromers (Ie-h) was converted to its corresponding benzyl ester p-toluene sulphonate salts (IIa-h.p-TsOH) by treatment with benzyl alcohol and p-toluene sulphonic acid monohydrate by refluxing in cyclohexane and simultaneously removing the water formed during reaction by azeotropic distillation. The mixture of p-toluene sulphonic acid salts of benzyl esters (IIa-IIj). p-TsOH was then purified by crystallization from various solvents selected from cyclohexane, dichloromethane, ethyl acetate and diisopropyl ether or mixtures thereof, preferably from a mixture of dichloromethane-cyclohexane dichloromethane-diisopropyl ether or ethyl acetate-diisopropyl ether. A comparison of purity and yield obtained by using various solvents for crystallization is indicated in Table 1.

TABLE 1
Enrichment of purity by crystallization of mixture of benzyl ester
p-toluene sulphonate salts (IIa-h.p-TsOH) from various solvents.

| | | Ratio of | Purity | |
|---|---|---|---|---|
| Sr. | | solvent | obtained | Yield |
| No. | Solvent | (v/v) | (%) | (%) |
| 1 | Cyclohexane | — | 98.2 | 95 |
| 2 | Dichloromethane-Cyclohexane | 2:6 | 99.89 | 62 |
| 3 | Dichloromethane-Cyclohexane | 1:5 | 99.27 | 94 |
| 4 | Dichloromethane-Cyclohexane | 1.5:5 | 99.72 | 95 |
| 5 | Dichloromethane-Cyclohexane | 1.5:5 | 99.44 | 89 |
| 6 | Dichloromethane-Diisopropyl ether | 2:5 | 98.47 | 90 |
| 7 | Ethyl acetate-Cyclohexane | 1:5 | 99.25 | 96 |

The invention involves the appropriate selection of solvent for purification and to provide a process for obtaining the mixture of p-toluene sulphonic acid salts of benzyl esters IIa and IIb in a purity >99%.

The conversion of salts IIa.p-TsOH and IIb.p-TsOH to free esters (IIa and IIb) has been achieved by treatment with inorganic bases such as sodium carbonate, sodium bicarbonate, potassium carbonate, potassium bicarbonate, sodium hydroxide, potassium hydroxide etc in biphasic mixture containing water immiscible organic solvent such as ethyl acetate, dichloromethane and water at lower temperature such as 0-10° C., preferably 0-5° C.

The resolution of the racemic mixture of benzyl esters IIa and IIb with DBTA was accomplished in various solvents such as ethanol, methanol, acetonitrile, ethyl acetate, acetone mixture of dimethyl sulphoxide and acetonitrile, mixture of dimethyl formamide and acetonitrile. The chiral purity and yield obtained in different solvents is indicated in Table 2. The preferred solvent for resolution is mixture of dimethyl formamide-acetonitrile or dimethyl sulphoxide-acetonitrile. The most preferred solvent is mixture of dimethyl formamide-acetonitrile.

TABLE 2
Resolution of racemic benzyl esters IIa and IIb in various solvents.

| | | | Ratio of IIa:IIb | | |
|---|---|---|---|---|---|
| | | | (% by HPLC on | | |
| Sr. | | Volume of | chiral column) | | Yield of |
| No. | Solvent | solvent | IIa | IIb | IIa. DBTA (%) |
| 1 | Ethanol | 7 | 87.5 | 12.5 | * |
| 2 | Methanol | 15 | 97.1 | 2.9 | 50.6 |
| 3 | Acetonitrile | 35 | 52.3 | 47.7 | No resolution |
| 4 | Ethyl acetate | 10 | 51.9 | 48.1 | No resolution |
| 5 | Acetone | 8 | 59.5 | 40.5 | No resolution |
| 6 | Dimethyl sulphoxide-acetonitrile | 20 | 99.4 | 0.6 | 38.6 |
| | (20:80) | | | | |
| 7 | Dimethyl formamide-acetonitrile | 25 | 98.5 | 1.5 | 64 |
| | (30:70) | | | | |

* This crude product on further recrystallisation from ethanol afforded pure IIa. DBTA salt in 99.4% chiral purity and 64% yield.

In a preferred aspect the resolution of the racemic mixture of benzyl esters IIa and IIb was carried out with DBTA in a mixture of dimethyl formamide and acetonitrile at temperature between 15° C. to 35° C. When resolution was carried out at 25-35° C. impurity formation was up to 2-3.6%. In a further preferred aspect the resolution carried out at 15-20° C. in which the unknown impurity formation was controlled below 2%. The effect of variation in ratio of dimethyl formamide to acetonitrile is shown in table 3.

TABLE 3
Effect of variation in ratio of dimethyl formamide
and acetonitrile in resolution of IIa and IIb

| | | | | Ratio of IIa:IIb | | |
|---|---|---|---|---|---|---|
| | | | | (% by HPLC | | |
| | Ratio of dimethyl formamide- | | | on chiral | | |
| Sr. | acetonitrile in the solvent | | Temperature | column) | | Yield of |
| No. | Dimethyl formamide | Acetonitrile | (° C.) | IIa | IIb | IIa. DBTA (%) |
| 1 | 20 | 80 | 25-30 | 75 | 25 | Poor resolution |
| 2 | 25 | 75 | 25-30 | 75.8 | 24.2 | Poor resolution |
| 3 | 30 | 70 | 25-30 | 98.5 | 1.5 | 64 |
| 4 | 35 | 65 | 25-30 | 98 | 2 | 64 |
| 5 | 40 | 60 | 25-30 | 97.7 | 2.2 | 41 |
| 6 | 45 | 55 | 25-30 | 98 | 1.9 | 33 |
| 7 | 50 | 50 | 25-30 | 95.8 | 4.1 | 11 |
| 8 | 30 | 70 | 15-18 | 97.4 | 2.5 | 72 |

The conversion of salt IIa.DBTA to free benzyl ester (IIa) has been achieved by treatment with inorganic bases such as sodium carbonate, sodium bicarbonate, potassium carbonate, potassium bicarbonate, sodium hydroxide, potassium hydroxide etc in biphasic mixture containing water immiscible organic solvent such as ethyl acetate, dichloromethane and water at lower temperature such as 0-10° C., preferably 0-5° C.

The optically pure enantiomer benzyl ester IIa is converted to trandolapril benzyl ester (IVa) by treating with NEPA-NCA (IIIb) in dichloromethane which on deprotection of the benzyl group by catalytic hydrogenation over Pd/C in ethanol furnished crude trandolapril.

The crude trandolapril is purified by recrystallization from solvents such as ethanol, mixture of ethanol-diisopropyl ether, ethyl acetate, acetone, methyl ethyl ketone, acetonitrile, tetrahydrofuran, nitromethane and dimethoxy propane. Among these preferred is a mixture of ethanol and diisopropyl ether. In a preferred embodiment the ratio 3:5 and 2:5 of ethanol and diisopropyl ether was studied. The preferred ratio is 2:5 in which purity >99.5% and yield >70% (from crude trandolapril) was obtained. The crystallization from ethanol-diisopropyl ether minimizes the formation of diketopiperazine impurity. Also it resulted in reduction of trandolapril analogues below 0.1% which were arising from cis endo ester (II) and unknown impurity formed by epimerisation in resolution. The results of crystallization of crude trandolapril are shown in table 4.

TABLE 4
Crystallization of crude trandolapril in various solvents.

| Sr. | | Assay by HPLC* | Yield |
|---|---|---|---|
| No. | Solvent | (%) | (%) |
| 1 | Ethyl acetate | 98.2 | 92 |
| 2 | Acetone | 98.7 | 76 |
| 3 | Methyl ethyl ketone | 98.8 | 84 |
| 4 | Acetonitrile | 98.6 | 81 |
| 5 | Tetrahydrofuran | 97.9 | 31 |
| 6 | Nitromethane | 96.3 | 65.5 |
| 7 | Dimethoxy propane | 97.7 | 74 |
| 8 | Ethanol | 98.6 | 85 |
| 9 | Ethanol-Diisopropyl ether (3:5) | 98 | 83 |
| 10 | Ethanol-Diisopropyl ether (2:5) | 99.3 | 89.2 |

*Isocratic system. Column: RP18 (150 × 4.6 mm), 4μ; Flow: 1.5 ml/minute; Detector: UV 210 nm; Buffer: 0.05 molar Na₂HPO₄ + triethylamine + acetonitrile (1500:3:555), pH adjusted to 1.5-2.5.

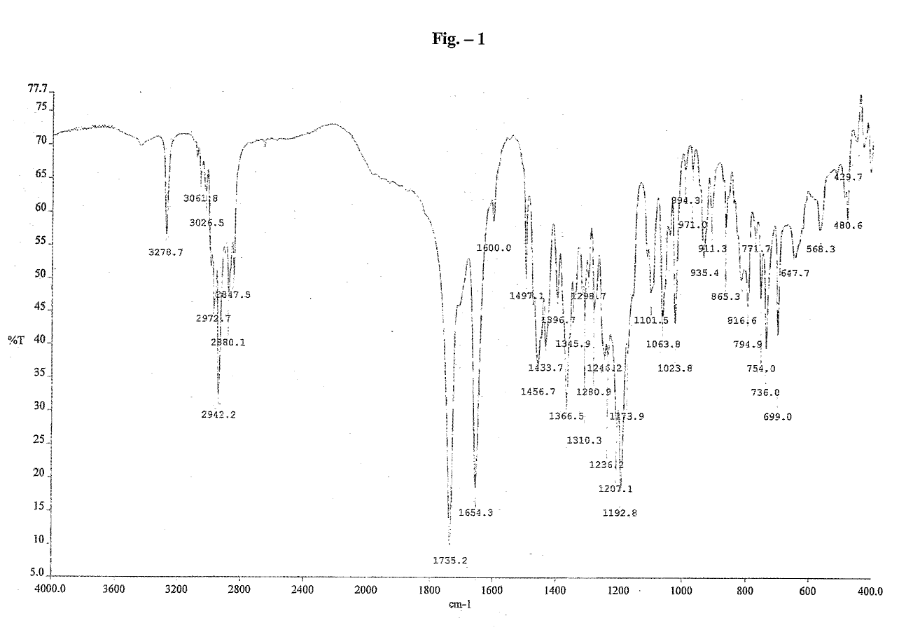

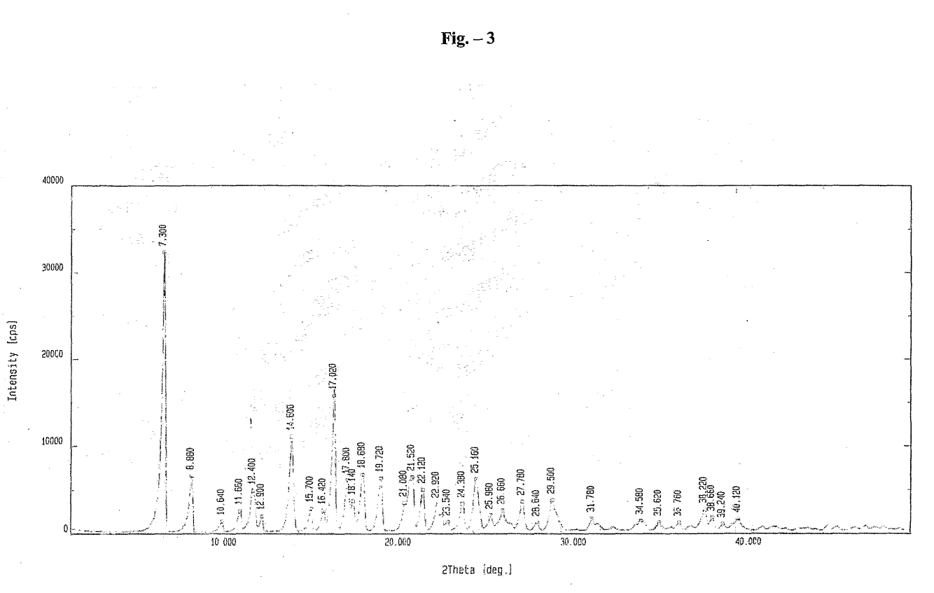

The infrared spectrum of crystallized trandolapril obtained by the process of the present invention is given in FIG. 1 and the characteristic X-ray powder diffraction pattern is given in FIG. 3.

Though in the example 42 (c) of the product U.S. Pat. No. 4,933,361 the nature of trandolapril is mentioned as foam, it was found that while repeating the same procedure and evaporating the solvent under reduced pressure (2-4 mm Hg) for longer time (20 hours) trandolapril as solid was obtained.

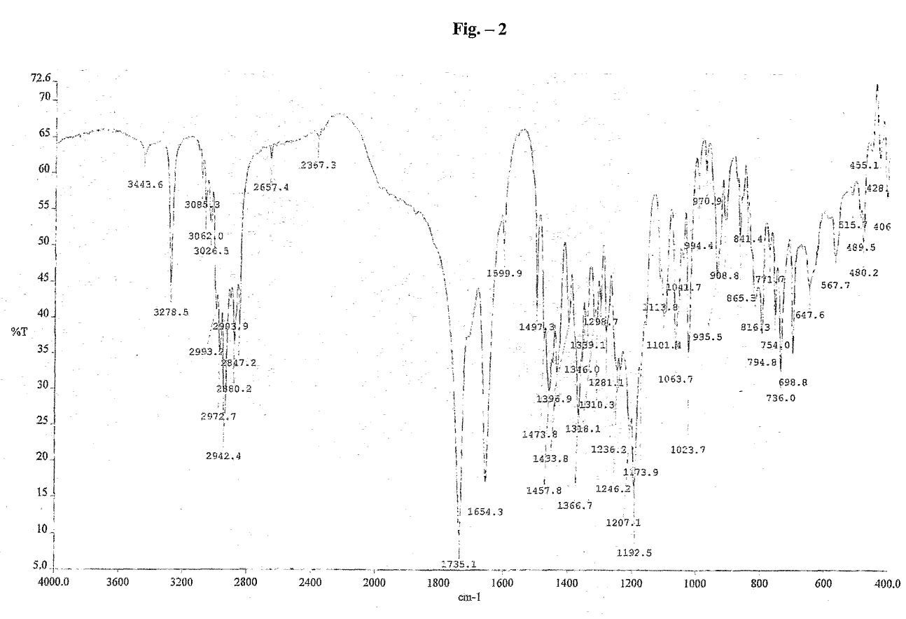

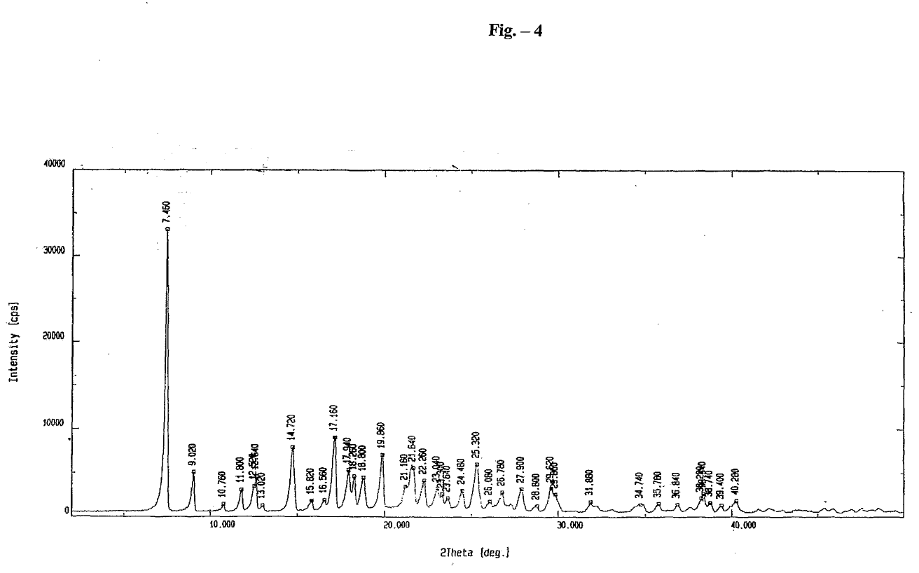

The infra red spectrum and X-ray powder diffraction pattern of trandolapril solid obtained by practicing the process disclosed in product U.S. Pat. No. 4,933,361 is given in FIG. 2 and FIG. 4 respectively.

The infrared spectrum crystallized trandolapril obtained by the process of the present invention (FIG. 1) and that of the product obtained by the process disclosed in the product patent US'361 shown in (FIG. 2) are identical.

The powder XRD of crystallized trandolapril obtained by the process of the present invention (FIG. 3) that of the product obtained by the process disclosed in the product patent US'361 shown in (FIG. 4) are also identical.

The invention is further illustrated by the following non-limiting examples.

### EXAMPLE 1

Step 1. Preparation of Benzyl Ester p-toluenesulphonate Salt (IIa-h.p-Ts-OH)

A mixture of racemic amino acid Ia-h (83 gm, 0.491 mole), p-toluenesulphonic acid monohydrate (186.6 gm, 0.982 moles), and benzyl alcohol (265.2 gm, 2.455 moles) in cyclohexane (830 ml), was slowly heated to reflux temperature (79-80° C.) for about 10-12 hours. The cyclohexane was distilled under reduced pressure till thick mobile residue was left. The residue was cooled to 25-30° C. and diisopropyl ether (2490 ml) was added. The white solid separated out was filtered, washed with diisopropyl ether (274 ml). Yield: 323.7 g (wet solid) and HPLC purity 94.9%.

Step 2. Purification of Benzyl Ester p-TsOH Salts (IIa-h.p-TsOH)

A flask was charged with dichloromethane (448.2 ml), wet solid benzyl ester p-TsOH salt (323.7 gm) obtained above in step 1 was added with stirring at 25-30° C. Cyclohexane (747 ml) was added to the slurry at 25-30° C. The reaction mixture was heated further to 50-55° C. Cyclohexane (747 ml) was added to the slurry and heating continued further at for 1 hour. The reaction mixture was then cooled to 25-30° C., filtered and the solid was washed with a mixture of dichloromethane (80 ml) and cyclohexane (280 ml). Solid dried under reduced pressure at 50-55° C. for 4-5 hours Yield: 257.3 gm and HPLC purity 99.1%.

Step 3. Preparation of Racemic Benzyl Ester (IIa+IIb) Free Base

Racemic benzyl ester p-TsOH salt (211.6 gm, 0.491 moles) obtained in step 2 above was added to flask containing dichloromethane (622.5 ml). Cooled to 0° C. A cooled aqueous solution of cold 5% sodium bicarbonate (2905 ml) was added maintaining the temperature below 5° C. Stirred at 2-5° C. for 15-20 minutes to get a clear biphasic mixture. The organic layer was separated and washed twice with 5% sodium bicarbonate solution (581 ml) followed by saturated sodium chloride solution (83 ml). The organic layer was concentrated under reduced pressure to give thick light brownish liquid. Yield 95.68 gm and HPLC purity 98.96%.

Step 4. Resolution of the Racemic Benzyl Esters IIa and IIb

The racemic benzyl ester IIa+IIb (41 gm, 0.158 mole) obtained in step 3 above was charged to flask containing acetonitrile (574 ml). Cooled to 15-20° C. and then dimethyl formamide (246 ml). A solution of (−)-dibenzoyl-L-tartaric acid monohydrate (61.29 gm, 0.163 mole) in mixture of acetonitrile (143.5 ml) and dimethyl formamide (61.5 ml) was slowly added at 15-20° C. Seed of salt IIa.DBTA (0.041 gm) was added. The resulting solution was stirred for 5 hrs at 15-20° C. The dibenzoyl tartarate salt of the benzyl ester IIa (IIa.DBTA) separated as solid was filtered and washed with acetonitrile (20.5). The solid was dried at 50-55° C. under reduced pressure for 10 hrs. Yield of IIa.DBTA was 28.7 gm and chiral purity by HPLC 98.18%.

Step 5. Preparation of Benzyl Ester IIa

The dibenzoyl tartarate salt IIa.DBTA (26 gm, 0.042 mole) obtained in step 4 above was charged into dichloromethane (130 ml), cooled to 0-2° C. An aqueous solution of cold 5% NaHCO₃ (260 ml) was added with maintaining the temperature 2-4° C. The organic layer was separated and washed twice with 5% NaHCO₃ (78 ml) followed by saturated sodium chloride solution (13 ml). The organic layer was concentrated under reduced pressure at 35-40° C. to give benzyl ester IIa as a thick gummy mass. Yield 10.87 gm and HPLC purity 98.06%. The ester IIa was converted to its hydrochloride salt and its specific optical rotation [α]_{D} of ester hydrochloride (IIa.HCl) checked which was −41.8° (c=0.5, acetone) [Lit. −43°]

Step 6. Preparation of Trandolapril Benzyl Ester (IVa)

Benzyl ester IIa (10.87 gm, 0.042 moles) obtained in step 5 above was dissolved in dichloromethane (40 ml) and cooled to 0-2° C. N-[1-(S)-ethoxycarbonyl-3-phenylpropyl]-(S)-alanine N-carboxy anhydride (NEPA-NCA, IIIa) (13.49 gm, 0.044 mole) was added and stirred at 2-3° C. for 2 hours. Solution of 5% sodium bicarbonate (130 ml) and triethyl amine (0.85 gm) was added and stirred for 19 hours. The layers were separated. The organic layer washed twice with 5% sodium bicarbonate (52 ml) followed by water (13 ml). The organic layer was concentrated under reduced pressure at 40-45° C. to get a gummy solid. Yield was 21.84 gm and HPLC purity 97.8%.

Step 7. Preparation of Crude Trandolapril

The gummy mass of trandolapril benzyl ester IVa (21.84 gm, 0.042 mole) obtained above in step 6 was dissolved in ethanol (410 ml) at 25-30° C. and charged to autoclave. 10% Pd/C (2.184 g) was added under nitrogen at 25-30° C. The reaction mixture was stirred at 25-30° C. for 2 hours maintaining the hydrogen pressure at 50 psi. The contents were filtered off, and catalyst washed with ethanol (60 ml). The combined filtrate was charged into another flask and ethanol was distilled off under reduced pressure at 35-40° C. till solid was left. Yield of crude trandolapril was 16.5 gm.

Step 8. Crystallization of Crude Trandolapril

Mixture of crude trandolapril (16.5 gm) obtained in step 7 above, ethanol (36.4 ml), and diisopropyl ether (91 ml) was refluxed for 10 minutes. Slowly cooled to 25° C. The solid obtained was filtered off, washed with diisopropyl ether (7.8 ml). Yield of pure trandolapril was 11.848 gm and HPLC purity 99.94% on gradient system and assay 99.2% (on gradient system).

M.P.: 122-124° C.,

IR (KBr): 3278.7, 2942.2, 1735.2, 1654.3, 1456.7, 1433.7, 1366.5, 1192.8, 1101.5, 1063.8 and 1023.8 cm⁻¹ (FIG. 1).

¹H NMR (CD₃OD, δ ppm): 7.33 (s, 5H), 4.34 (m, 3H), 3.86 (q, 2H), 3.28-1.46 (m, 17H) and 1.39 (d+t, 6H),

Mass (m/z, amu): 453.5 (M+Na) and 431.7 (M+H)⁺ molecular ion.

Powder XRD: The (d) spacings and relative intensities (I/Io) are listed below.

| d | Relative intensity (%) |
|---|---|
| 7.30 | 100 |
| 8.88 | 20 |
| 11.66 | 8 |
| 12.4 | 15 |
| 12.9 | 6 |
| 14.6 | 34 |
| 15.7 | 9 |
| 16.42 | 8 |
| 17.02 | 49 |
| 17.8 | 19 |
| 18.14 | 11 |
| 18.68 | 21 |
| 19.72 | 19 |
| 21.08 | 7 |
| 21.32 | 11 |
| 21.50 | 20 |
| 22.12 | 16 |
| 22.92 | 10 |
| 23.15 | 6 |
| 24.38 | 11 |
| 25.16 | 19 |
| 25.98 | 6 |
| 26.66 | 8 |
| 27.78 | 11 |
| 29.5 | 12 |
| 38.22 | 8 |

The crystalline trandolapril obtained by the above process of the present invention has the characteristic X-ray powder diffraction pattern as given in FIG. 3

### EXAMPLE 2

### Preparation of Trandolapril as Per Example 42 (c) Described in Product U.S. Pat. No. 4,933,361

The gummy mass of trandolapril benzyl ester IVa (42 gm, 0.0807 mole) was dissolved in ethanol (1482.3 ml) at 23° C. and solution was charged into autoclave. 10% Pd/C (4.94 gm) was added reaction mixture was hydrogenated under normal pressure at 23° C. for 2 hours. The contents were filtered and filtrate was evaporated to give foamy solid.

The resulting foamy solid was further concentrated under reduced pressure (2-4 mm Hg) for 5 hours to remove the traces of solvent. The trandolapril was obtained was further dried under reduced pressure (2-4 mm Hg) for 20 hours. Yield was 17.2 gm and HPLC purity 98.8%.

M.P.: 117.5-118.5° C.,

IR (KBr): 3278.5, 2942.4, 1735.1, 1654.3, 1457.8, 1433.8, 1366.7, 1192.5, 1101.4, 1063.7 and 1023.7 cm⁻¹ (FIG. 2)

Powder XRD: The (d) spacing and relative intensities (I/Io) are listed below.

| d | Relative intensity (%) |
|---|---|
| 7.46 | 100 |
| 9.02 | 15 |
| 11.8 | 9 |
| 12.52 | 10 |
| 12.64 | 14 |
| 14.72 | 24 |
| 15.82 | 5 |
| 16.56 | 6 |
| 17.16 | 27 |
| 17.94 | 16 |
| 18.26 | 14 |
| 18.80 | 14 |
| 19.86 | 21 |
| 21.18 | 10 |
| 21.64 | 17 |
| 22.26 | 13 |
| 23.04 | 10 |
| 23.30 | 8 |
| 23.64 | 7 |
| 24.48 | 9 |
| 25.32 | 18 |
| 26.08 | 5 |
| 26.78 | 9 |
| 27.90 | 10 |
| 29.62 | 10 |
| 38.34 | 8 |

The characteristic X-ray powder diffraction pattern of trandolapril obtained by the above example 2 is given in FIG. 4

1. A process for preparation of highly pure trandolapril of formula (1) 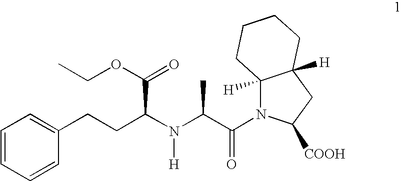
comprising the steps of:
(a) crystallization of mixture of crude octahydro benzyl esters (IIa-h.p-TsOH) to provide a racemic mixture of benzyl trans-(2S, 3aR, 7aS)-octahydro-1H-indole carboxylate p-toluene sulphonic acid salt (IIa.p-TsOH) and benzyl trans-(2R, 3aS, 7aR)-octahydro-1H-indole carboxylate p-toluene sulphonic acid salt (IIb.p-TsOH) of purity greater than 99% from dichloromethane, ethyl acetate, cyclohexane and diisopropyl ether or mixtures thereof, 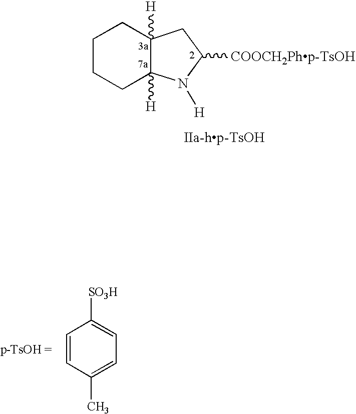
(b) conversion of racemic mixture of IIa.p-TsOH and IIb.p-TsOH obtained above in step (a) is converted to the corresponding racemic mixture of benzyl trans-(2S, 3aR, 7aS)-octahydro-1H-indole carboxylate (IIa) and benzyl trans-(2R, 3aS, 7aR)-octahydro-1H-indole carboxylate (IIb) by treatment with aqueous sodium bicarbonate in dichloromethane; 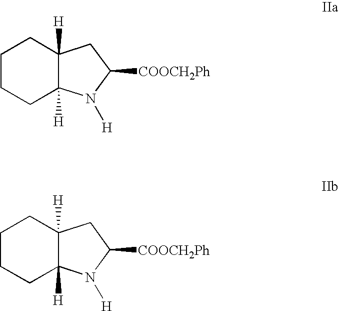
(c) optical resolution of racemic mixture of free benzyl esters Ia and IIb obtained in step (b) with (−)-dibenzoyl-L-tartaric acid monohydrate in aprotic solvent to provide benzyl trans-(2S, 3aR, 7aS)-octahydro-1H-indole carboxylate (−)-dibenzoyl-L-tartaric acid salt (IIa.DBTA); 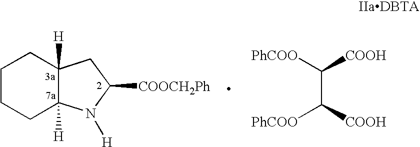
(d) conversion of salt IIa.DBTA obtained above in step (c) to free benzyl ester (IIa) by treatment with aqueous sodium bicarbonate in dichloromethane;
(e) reacting free benzyl ester (IIa) obtained in step (d) with N-[1-(S)-ethoxycarbonyl-3-phenylpropyl]-(S)-alanine N-carboxy anhydride (IIa, NEPA-NCA) to get trandolapril benzyl ester (IVa), 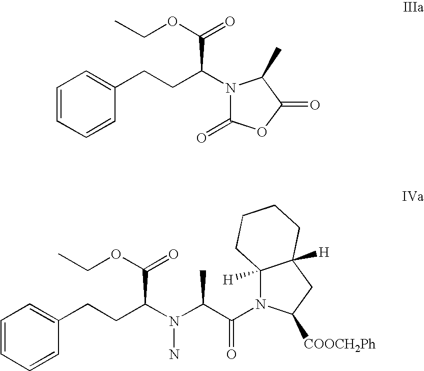
(g) hydrogenolysis of the trandolapril benzyl ester (IVa) obtained in step (e) to get crude trandolapril
and
(g) crystallization of crude trandolapril obtained in step (f) from mixture of ethanol and diisopropyl ether.

2. A process according to claim 1, wherein the step (a) comprises of:
(a) heating IIa-h.p-TsOH salts in a mixture of organic solvent of first type and organic solvent of second type, or heating in organic solvent of the first type and adding the organic solvent of the second type during heating,
(b) refluxing the mixture,
(c) cooling and isolating the solid by filtration

3. A process according to claim 2, wherein the organic solvent of first type is selected from dichloromethane, ethyl acetate and cyclohexane or mixtures thereof, preferably dichloromethane.

4. A process according to claim 2, wherein the organic solvent of second type is cyclohexane and diisopropyl ether, preferably cyclohexane.

5. A process according to claim 2, wherein the ratio of first type of organic solvent to second type of organic solvent varies from 100:0 to 0:100, preferably 1:2 to 1:6, most preferably 1:3 to 1:5.

6. A process according to claim 2, wherein the reflux temperature is between 60-80° C.

7. A process according to claim 2, wherein the mixture is cooled to 25-30° C.

8. A process according to claim 1, wherein the step (b) at temperature 0-40° C., preferably at 0-10° C.

9. A process according to claim 1, wherein the step (c) comprises of:
(a) providing a solution of racemic mixture of IIa and IIb in a mixture of aprotic solvents,
(b) cooling the solution,
(c) adding a solution of DBTA in mixture of aprotic solvents,
(d) mixing of DBTA solution with cold solution of esters IIa and IIb at lower temperature,
(e) optionally seeding with salt IIa.DBTA,
(f) stirring at lower temperature to crystallize DBTA salt of pure enantiomer Ia (IIa.DBTA) and
(g) isolating solid by filtration and washing of salt (IIa.DBTA) with aprotic solvent.

10. A process according to claim 9, wherein (−)-dibenzoyl-L-tartaric acid monohydrate (DBTA) is 0.9 to 1.2 mole equivalent preferably 1.0 to 1.1 equivalent.

11. A process according to claim 9, wherein the aprotic solvent is selected from acetonitrile, dimethyl sulfoxide, and dimethyl formamide or mixtures thereof, preferably mixture of dimethyl formamide and acetonitrile.

12. A process according to claim 9, wherein the mixing of DBTA solution to solution of esters IIa and IIb is carried out at 0-50° C., preferably at 10-20° C.,

13. A process according to claim 9, wherein the optical resolution is carried out at 0-50° C., preferably at 10-20° C.,

14. A process according to claim 9, wherein the aprotic solvent is mixture of dimethyl formamide and acetonitrile.

15. A process according to claim 14, wherein the ratio of dimethyl formamide-acetonitrile is in the range between 10:90 to 90:10, preferably 30:70.

16. A process according to claim 1, wherein the step (d) is carried out at temperature 0-40° C., preferably at 0-10° C.

17. A process according to claim 1, wherein the step (e) is carried out in organic solvent such as dichloromethane containing organic base such as triethyl amine at temperature between 0-40° C., preferably between 0-10° C.

18. A process according to claim 1, wherein the step (f) is carried out in ethanol in presence of 10% Pd/C under hydrogen pressure at 20-40° C., preferably at 25-30° C.

19. A process according to claim 1, wherein the step (g) is carried out in organic solvent consisting of ethanol, diisopropyl ether, acetone, methyl ethyl ketone ethyl acetate, tetrahydrofuran, acetonitrile, nitro methane or mixtures there of, preferably mixture of ethanol, diisopropyl ether.

20. A process according to claim 19, wherein ethanol-diisopropyl ether are in the ratio 1:9 to 9:1, preferably 1:1 to 1:3.
